(19) United States
(12) Patent Application Publication
(10) Pub. No.: US 2018/0188182 A1
(43) Pub. Date: Jul. 5, 2018

(54) FOOD PRODUCT SPOILAGE DETECTION

(71) Applicant: Wal-Mart Stores, Inc., Bentonville, AR (US)

(72) Inventors: Jacob Avery Jones, Centerton, AR (US); Kevin Matthew Charles, Bentonville, AR (US); Joshua Freeman, Bentonville, AR (US)

(21) Appl. No.: 15/856,203

(22) Filed: Dec. 28, 2017

Related U.S. Application Data
(60) Provisional application No. 62/440,854, filed on Dec. 30, 2016.

(51) Int. Cl. G01N 21/78; G01N 33/04; B65D 85/80; B65D 51/24

(52) U.S. Cl. CPC G01N 21/783; G01N 33/04; G06K 19/06028; B65D 51/245; B65D 85/80

(57) ABSTRACT

A container cap comprises a first layer that includes a material that changes color or opacity in response to a chemical reaction at the first layer between a gas emitted by a source of perishable food item in the container and the material of the first layer; a second layer on top of the first layer that includes a viewing window that displays the color or opacity of the material of the first layer; and an identifying marker revealed via the second layer in response to the first layer changing color or opacity indicative of the perishable food item being spoiled.

### RELATED APPLICATIONS

This application claims the benefit of U.S. Provisional Patent Application No. 62/440,854, filed Dec. 30, 2016 and entitled “Dairy Product Spoilage Detection”, the contents of which are incorporated herein in their entirety.

### TECHNICAL FIELD

The present inventive concepts relate generally to the spoilage of perishable foods, and more specifically, to systems and methods for detecting and analyzing changes in a stored food product.

### BACKGROUND

A viewer of a milk container cannot readily discern the freshness of the milk inside the container without opening the container and smelling its contents. Retail establishments that sell dairy products desire an efficient approach for determining whether certain containers include spoiled milk or the like. Also, retail establishments desire to simplify a store associate's job when identifying particular food containers for spoilage.

### SUMMARY

In one aspect, provided is a container cap for coupling to a container, comprising: a first layer that includes a material that changes color or opacity in response to a chemical reaction at the first layer between a gas emitted by a source of perishable food item in the container and the material of the first layer; a second layer on top of the first layer that includes a viewing window that displays the color or opacity of the material of the first layer; and an identifying marker revealed via the second layer in response to the first layer changing color or opacity indicative of the perishable food item being spoiled.

In some embodiments, the perishable food item includes milk.

In some embodiments, the container cap further comprises a third layer below the first and second layers that includes a semi-permeable, micromesh material for directing the gas emitted by the source of perishable food item from the container to the first layer of the cap.

In some embodiments, the third layer prevents liquid of the source of perishable food item from contacting the material of the first layer, while allowing the gas emitted by the source of perishable food item to be received by the material.

In some embodiments, the material of the first layer in response to the chemical reaction changes from a first color to a second color when the detected gas corresponds to the source of perishable food item that is beginning to expire, and wherein the first layer changes from the second color to a third color when the detected gas corresponds to source of perishable food item that has expired.

In some embodiments, the identifying marker includes a barcode or a QR code.

In some embodiments, the identifying marker is positioned under the first layer, which changes in opacity so that the identifying marker can be viewed through the first layer.

In some embodiments, the identifying marker is integrated with the first layer, and is revealed when the first layer changes color.

In some embodiments, the identifying marker is not viewable from the second layer when the perishable food item is fresh.

In some embodiments, the perishable food item includes a carbonated beverage, and the identifying marker establishes when the carbonated beverage does not include a predetermined amount of carbon dioxide.

In some embodiments, the perishable food item is prone to fermentation, and the identifying marker establishes when the perishable food item is fermented.

In another aspect, provided is a container cap for coupling to a container, comprising a first layer that includes a material that changes color in response to a chemical reaction at the first layer between a gas emitted by a source of perishable food item in the container and the material of the first layer; a second layer below the first layer that includes a semi-permeable, micromesh material for directing the gas emitted by the source of perishable food item from the container to the first layer of the cap; and an identifying marker revealed when the perishable food item is spoiled.

In some embodiments, the container cap further comprises a third layer on top of the first layer that includes a viewing window that displays the color of the material of the first layer.

In some embodiments, the second layer prevents liquid of the source of perishable food item from contacting the material of the first layer, while allowing the gas emitted by the source of perishable food item to be received by the material.

In some embodiments, the identifying marker includes a barcode or a QR code.

In another aspect, provided is a spoilage detection system, comprising: a container element that includes an identifying marker that is visible to a scanning device when a material of the cap changes from a first state to a second state when a perishable food item proximal the container element turns from a fresh condition to a spoiled condition; at least one sensor that provides an input regarding an environment in which the perishable food item is located; and a spoilage analytics system that collects a combination of real-time, near real-time, and historical input data from the identifying marker and the at least one sensor to generate a result regarding a cause of the change from the fresh condition to the spoiled condition.

In some embodiments, the container element comprises: a first layer that includes a material that changes color in response to a chemical reaction at the first layer between a gas emitted by a source of perishable food item in the container and the material of the first layer; a second layer below the first layer that includes a semi-permeable, micromesh material for directing the gas emitted by the source of perishable food item from the container to the first layer of the cap; and the identifying marker revealed when the perishable food item is spoiled.

### BRIEF DESCRIPTION OF THE DRAWINGS

FIG. 1 is a perspective view of a dairy container having a closure cap constructed and arranged according to some embodiments.

FIG. 2 is a cross-sectional blowup view of the container closure cap of FIG. 1

FIG. 3 is an exploded view of the container closure cap of FIGS. 1 and 2.

FIG. 4A is a top view of the container closure cap of FIGS. 1-3 in a first state.

FIG. 4B is a top view of the container closure cap of FIGS. 1-4A in a second state.

FIG. 5 is a schematic of an environment in which a spoilage analytics system may be implemented, in accordance with some embodiments.

### DETAILED DESCRIPTION OF EMBODIMENTS

In brief overview, a portion of a container such as a closure cap is constructed and arranged to detect gases released by a perishable item, such as milk or other dairy product stored in the container, or other products such as soda products. In doing so, the closure cap or the like can indicate a state of the dairy product, for example, by a color code indicating whether the dairy product in the container is fresh, nearly expired, or spoiled. Thus, a viewer of the container can immediately determine from the color of the closure cap whether the dairy product inside the container is spoiled without opening the container.

In some embodiments, the closure cap may present for display an identifying marker such as a barcode, QR code, or the like that includes data that may be processed by an analytics system and/or other sensors that collect input regarding the environment in which the container is located. For example, a temperature sensor can provide temperature information to the analytics system that combines the temperature information with a barcode result indicating that several containers of milk are spoiled to establish that the temperature at a dairy aisle where the containers are located needs to be decreased to reduce the amount of spoilage. A color state of the status indicator may inform a viewer such as a shopper or store associate the condition of the container contents. For example, the status indicator can change to a different color when the state of the dairy item changes from fresh to spoiled. Also, the identifying marker in some embodiments is only displayed when spoilage has been detected. For example, a barcode may be hidden or otherwise obscured by a cap having a dark color which “blends in” with the dark colored cap. However, the barcode may be revealed or otherwise exposed for display for reading by a scanner when the status indicator changes from a dark color to a light color so that the light colored background of the cap permits the barcode to be displayed for viewing and scanning. In another example, the status indicator may change in opacity, for example, change from an opaque layer to a translucent or clear layer so that a barcode under the layer may be viewed. When the barcode is exposed, a scanner can collect data from the barcode, which can be used to establish a time when the spoilage occurred or other condition-related information.

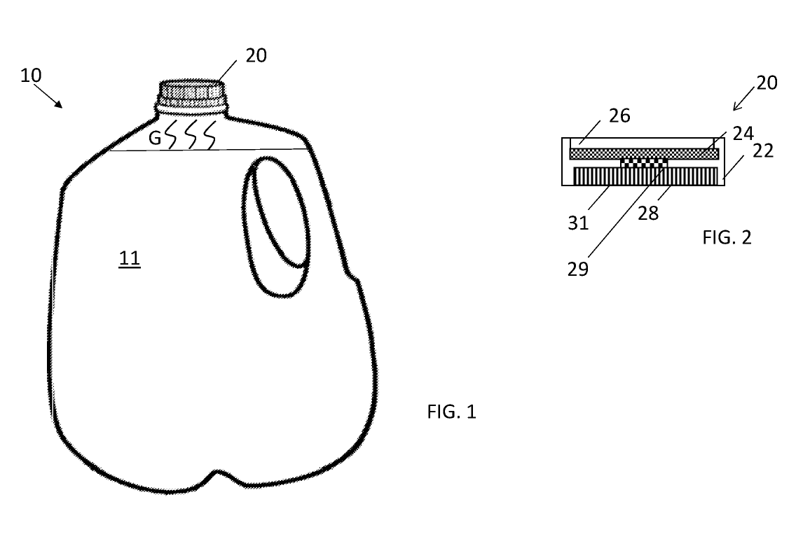

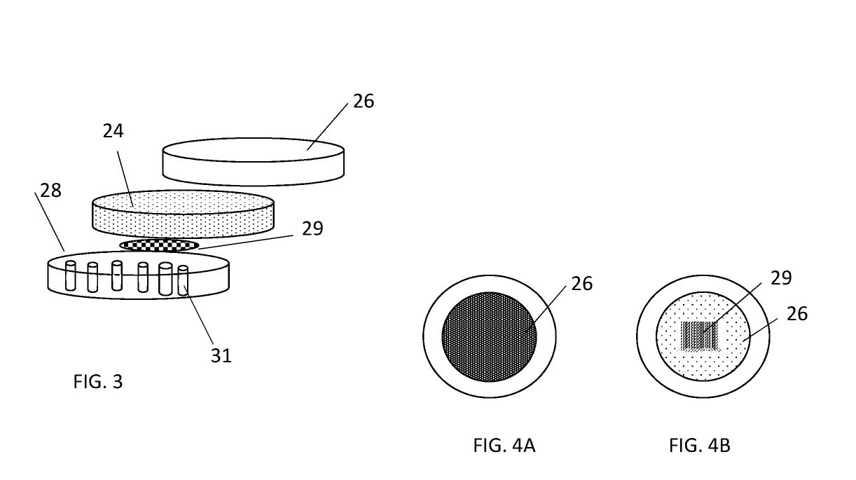

FIG. 1 is a perspective view of a dairy container 10 having a closure cap 20 constructed and arranged according to some embodiments. FIG. 2 is a cross-sectional blowup view of the container closure cap 20 of FIG. 1. FIG. 3 is an exploded view of the container closure cap 20 of FIGS. 1 and 2.

Although the container 10 shown in FIG. 1 is a gallon milk container, the closure cap 20 may be constructed and arranged on any container that holds perishable food items, for example, cardboard or plastic cartons, bottles, or other container having an opening configured for being covered with a closure cap. The closure cap 20 is not limited to containers, and in some embodiments is integrated with or otherwise retrofitted into an apparatus that communicates with a food product, for example, a device inserted into a refrigerator, pantry, or other storage location.

As shown in FIGS. 1-3, the closure cap 20 may include a housing 22 that partially or completely surrounds a multi-layer spoiler indicator comprising a first layer 24 sandwiched between a second layer 26 and a third layer 28. Although the spoiler indicator is shown and described with respect to a closure cap 20, in other embodiments, the multi-layer spoiler indicator comprising a first, second, and third layers 24, 26, 28, respectively, may be positioned at other regions of a container holding milk or other dairy products, for example, integrated into a sidewall, handle, or bottom of the container 10.

In some embodiments, the first layer 24 includes an impregnatable material that changes color in response to a chemical reaction at the first layer between a gas (G) emitted by a source of perishable food product 11 such as milk in the container 10 and the material of the first layer 24. In some embodiments, the first layer 24 includes phenolphthalein that reacts when milk becomes more acidic, i.e., forms lactic acid. In other embodiments, the first layer 24 may include a red cabbage layer. In other embodiments, other chemicals, acid-base indicators, or the like may be used to change the color or opacity of the first layer 24. The first layer 24 may detects parts per million (ppm) of the gas G emitted by the source of perishable food product 11. The material may be a gas-sensitive material. The material may also change colors and or opacity, for example, from opaque to translucent or clear, when exposed to gas indicative of a spoiled or expired food product. For example, the material of the first layer 24 may change color to reflect the freshness of the milk inside the container 10. For example, the first layer 24 may show a green color for fresh, unspoiled milk, and change to one or more different colors depending on the freshness of the product, for example, red color for spoiled milk.

To achieve a change in color in this manner, the material of the first layer 24 may be coated or otherwise include a non-toxic chemical such as a fabric material impregnated with phenolphthalein, and/or other chemicals, acid-base indicators, or the like for changing the color and/or opacity of the first layer 24. In some cases, the interaction between the emitted gases, acids, and so on from the spoiled food product and the chemical of the first layer material may cause the food product itself to change color, for example, due to fermentation producing carbon dioxide and/or other gases.

For example, the closure cap 20 may be used for covering a carbonated beverage. Here, the cap 20 can detect whether the carbonated beverage inside the bottle emits a source of carbon dioxide, i.e., to determine whether the carbonated beverage is “flat.” In another example, the cap 20 can detect fermentation in a bottle of grape juice. In these examples, the cap 20 may change color similar to the example herein with respect to spoiled milk.

In some embodiments, the first layer 24 is configured to receive a source of user-determined gas-sensitive material that is applied to a neutral surface of the first layer 24. For example, a user may apply a chemical spray directly to the first layer 24 so that the first layer 24 can function to detect a gas of interest. In this example, a user may desire to detect milk spoilage, and apply a source of phenolphthalein to the first layer 24 so that the first layer 24 can change colors and so on. Alternatively, the user may instead desire to detect the state of a carbonated beverage instead of milk and therefore apply a different chemical, such as a source of calcium hydroxide, to the first layer 24.

The second layer 26 is positioned on top of the first layer 24 and includes a viewing window that displays the color of the material of the first layer 24. The second layer 26 may be formed of clear plastic or other material that permits a viewer to see through the second layer 26 to the first layer 24. Therefore, any color or change in color of the first layer 24 may be observed.

Also, an identifying marker 29 such as a bar code, quick response (QR) code, or the like that is incorporated into the closure cap 20, for example, formed or otherwise presented at, above, or below the first layer 24 may likewise be observed via the clear window of the second layer 26. For example, the marker 29 may be constructed and arranged as bar code identification strip, similar to the barcodes used at supermarket checkout stands.

The third layer 28 may be formed of a semi-permeable, micromesh or other porous material, or a material having holes, micro apertures or the like that exposes the first layer 24 to gases or other emissions from a source of product in the container 10, and for directing a gas emitted by the source of perishable product from the container 10 to the first layer 24.

The third layer 28 may serve as a barrier for preventing chemical contamination of the dairy product in the container 10 from the first layer 24. In particular, the third layer 28 prevents liquid of the source of perishable dairy product from contacting the material of the first layer 24, while allowing the gas emitted by the source of perishable product to be received by the material 24. To porous feature of the third layer 28 prevents a leak of chemical from the first layer 24 into the food product in the container 10 since the chemical in the first layer 24 above the third layer 28 may be bonded during a fabrication process, e.g., printing process, and is therefore not a loose liquid that may “leak” into the food product below the third layer 28. The configuration and structure allows for the reduction in manufacturing costs as compared to other detection solutions.

In further describing the identifying marker 29, as shown in FIG. 4B, the identifying marker 29 may be revealed to a viewer when the product is spoiled, fermented, flat, or otherwise at or near an expiration. As shown in FIG. 4B, the identifying marker 29 is displayed when the first layer 24 is of a color indicative of the presence of spoiled, fermented, flat, or otherwise change in product freshness in the container 10. As shown in FIG. 4A, the identifying marker 29 is hidden from the viewer due to a color of the first layer 24 indicating the presence of fresh product. For example, as the gas or detectable acid emitted from the food product approaches the ink from which the identifying marker 29 is formed, the chemical which is invisible at the time can change to a different color.

The identifying marker 29 includes data that identifies the container 10 and/or its contents, so that when the marker 29 is read by a reader, scanning device, or the like, the container 10 can be tracked or otherwise identified.

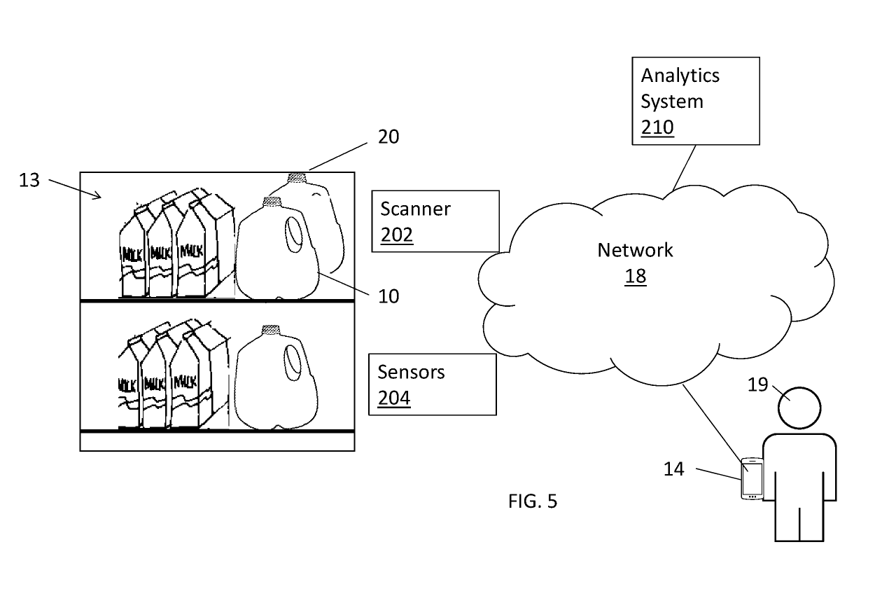

FIG. 5 is a schematic of an environment in which a spoilage analytics system 210 may be implemented, in accordance with some embodiments.

The spoilage analytics system 210 may collect a combination of real-time, near real-time, and historical input data from various data sources, create useful information from that data by filtering and isolating relevant data and performing analytics on the data, display that result data to a user 19 in an easy to understand format, and allow the user to interact with the displayed data via a personal computing device 14 such as a smartphone.

To perform the foregoing, the spoilage analytics system 210 is constructed and arranged to receive as inputs data from one or more sensors 204, which may include but not be limited to temperature meters, cameras, door sensors, and so on. For example, a door sensor may detect when a refrigerator door is open, indicative of reduced refrigeration conditions. The analytics system 210 may include artificial intelligence processing technology or other components that concludes that the open refrigerator door is the cause of spoiled milk containers on the shelves.

In another example, a voltage meter at the refrigeration area 13 may provide information regarding conditions when one or more containers 10 of milk went bad. In this example, a cap 20 of a container 10 may display a barcode 29, indicating that this container is spoiled. The cap 20 may also display a color such as red indicating that the milk is spoiled, for example, shown in FIG. 4B. Since the barcode 29 is visible due to the spoilage, a scanning device 202 can be used to scan the barcode to ascertain an identity and/or other information regarding the container 10 and its milk content, which may be used to remove the container from a store shelf or other location. This data along with data collected by the voltage meter sensor 204 can establish the power consumed by the refrigeration area 13 when the container of milk became spoiled. The scanning device 202 may automatically scan the visible barcode 29. For example, a freezer in the refrigeration area 13 may include sensors and/or scanners, which scan the barcode 29 automatically. Alternatively, a store associate may be equipped with a scanning device to scan the barcode 29 to automatically remove the container from the system for inventory purposes.

In another example, a camera can provide visual tracking information to establish where the container 10 was located at the time that the scanner 202 scanned the cap 20.

A personal computing device 14 may be used by a user 11 to retrieve an output from the spoilage analytics system 20, for example, a determination as to the conditions, cause, or other reason why a spoilage event occurred.

As described herein, some or all of the systems and methods in accordance with some embodiments are implemented in a computer system. The computer system may generally comprise a processor, an input device coupled to the processor, an output device coupled to the processor, and memory devices coupled to the processor via a bus or other signal-carrying connector. The processor may perform computations and control the functions of a computer, including executing instructions included in computer code for the tools and programs capable of implementing a method in the manner prescribed by the embodiments of the figures using the system described with respect to the figures, wherein the instructions of the computer code may be executed by processor via memory device. The computer code may include software or program instructions that may implement one or more algorithms for implementing the systems and methods, as described in detail above. The processor may execute the computer code.

A memory device may include input data. The input data includes any inputs required by the computer code. The output device may display output from the computer code. The memory device may be used as a computer usable storage medium (or program storage device) having a computer readable program embodied therein and/or having other data stored therein, wherein the computer readable program comprises the computer code. Generally, a computer program product (or, alternatively, an article of manufacture) of the computer system may comprise said computer usable storage medium (or said program storage device).

Memory devices include any known computer readable storage medium, including those described in detail below. In one embodiment, cache memory elements of memory devices may provide temporary storage of at least some program code in order to reduce the number of times code must be retrieved from bulk storage while instructions of the computer code are executed. Moreover, similar to processor, memory device may reside at a single physical location, including one or more types of data storage, or be distributed across a plurality of physical systems in various forms. Further, memory device can include data distributed across, for example, a local area network (LAN) or a wide area network (WAN). Further, memory device may include an operating system (not shown) and may include other systems not shown.

As will be appreciated by one skilled in the art, in a first embodiment, the present invention may be a method; in a second embodiment, the present invention may be a system; and in a third embodiment, the present invention may be a computer program product. Any of the components of the embodiments of the present invention can be deployed, managed, serviced, etc. by a service provider that offers to deploy or integrate computing infrastructure with respect to embodiments of the present inventive concepts. Thus, an embodiment of the present invention discloses a process for supporting computer infrastructure, where the process includes providing at least one support service for at least one of integrating, hosting, maintaining and deploying computer-readable code in a computer system including one or more processor(s), wherein the processor(s) carry out instructions contained in the computer code causing the computer system to allow an employment and operation of embodiments of the present invention. Another embodiment discloses a process for supporting computer infrastructure, where the process includes integrating computer-readable program code into a computer system including a processor.

The step of integrating includes storing the program code in a computer-readable storage device of the computer system through use of the processor. The program code, upon being executed by the processor, implements a method according to embodiments herein. Thus, the present invention discloses a process for supporting, deploying and/or integrating computer infrastructure, integrating, hosting, maintaining, and deploying computer-readable code into the computer system, wherein the code in combination with the computer system is capable of performing a method according to some embodiments.

A computer program product of the present invention comprises one or more computer readable hardware storage devices having computer readable program code stored therein, said program code containing instructions executable by one or more processors of a computer system to implement the methods of the present invention.

A computer system of the present invention comprises one or more processors, one or more memories, and one or more computer readable hardware storage devices, said one or more hardware storage devices containing program code executable by the one or more processors via the one or more memories to implement the methods of the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A container cap for coupling to a container, comprising:
a first layer that includes a material that changes color or opacity in response to a chemical reaction at the first layer between a gas emitted by a source of perishable food item in the container and the material of the first layer;
a second layer on top of the first layer that includes a viewing window that displays the color or opacity of the material of the first layer; and
an identifying marker viewable from the second layer via the second layer in response to the first layer changing color or opacity indicative of the perishable food item being spoiled.

2. The container cap of claim 1, wherein the perishable food item includes milk.

3. The container cap of claim 1, further comprising a third layer below the first and second layers that includes a semi-permeable, micromesh material for directing the gas emitted by the source of perishable food item from the container to the first layer of the cap.

4. The container cap of claim 3, wherein the third layer prevents liquid of the source of perishable food item from contacting the material of the first layer, while allowing the gas emitted by the source of perishable food item to be received by the material.

5. The container cap of claim 1, wherein the material of the first layer in response to the chemical reaction changes from a first color to a second color when the detected gas corresponds to the source of perishable food item that is beginning to expire, and wherein the first layer changes from the second color to a third color when the detected gas corresponds to source of perishable food item that has expired.

6. The container cap of claim 1, wherein the identifying marker includes a barcode or a QR code.

7. The container cap of claim 1, wherein the identifying marker is positioned under the first layer, which changes in opacity so that the identifying marker can be viewed through the first layer.

8. The container cap of claim 1, wherein the identifying marker is integrated with the first layer, and is revealed when the first layer changes color.

9. The container cap of claim 1, wherein the identifying marker is not viewable from the second layer when the perishable food item is fresh.

10. The container cap of claim 1, wherein the perishable food item includes a carbonated beverage, and wherein the identifying marker establishes when the carbonated beverage does not include a predetermined amount of carbon dioxide.

11. The container cap of claim 1, wherein the perishable food item is prone to fermentation, and wherein the identifying marker establishes when the perishable food item is fermented.

12. A container cap for coupling to a container, comprising:
a first layer that includes a material that changes color in response to a chemical reaction at the first layer between a gas emitted by a source of perishable food item in the container and the material of the first layer;
a second layer below the first layer that includes a semi-permeable, micromesh material for directing the gas emitted by the source of perishable food item from the container to the first layer of the cap; and
an identifying marker revealed when the perishable food item is spoiled.

13. The container cap of claim 12, further comprising a third layer on top of the first layer that includes a viewing window that displays the color of the material of the first layer.

14. The container cap of claim 12, wherein the second layer prevents liquid of the source of perishable food item from contacting the material of the first layer, while allowing the gas emitted by the source of perishable food item to be received by the material.

15. The container cap of claim 12, wherein the identifying marker includes a barcode or a QR code.

16. A spoilage detection system, comprising:
a container element that includes an identifying marker that is visible to a scanning device when a material of the cap changes from a first state to a second state when a perishable food item proximal the container element turns from a fresh condition to a spoiled condition;
at least one sensor that provides an input regarding an environment in which the perishable food item is located; and
a spoilage analytics system that collects a combination of real-time, near real-time, and historical input data from the identifying marker and the at least one sensor to generate a result regarding a cause of the change from the fresh condition to the spoiled condition.

17. The spoilage detection system of claim 16, wherein the container element comprises:
a first layer that includes a material that changes color in response to a chemical reaction at the first layer between a gas emitted by a source of perishable food item in the container and the material of the first layer; and
a second layer below the first layer that includes a semi-permeable, micromesh material for directing the gas emitted by the source of perishable food item from the container to the first layer of the cap, the identifying marker revealed when the perishable food item is spoiled.
